(19) United States
(12) Patent Application Publication
(10) Pub. No.: US 2013/0330423 A1
(43) Pub. Date: Dec. 12, 2013

(54) COMPOSITION TO INCREASE JOINT AND/OR POSTURAL STABILITY

(71) Applicant: Claudia Scholz, Darmstadt (DE)

(72) Inventor: Claudia Scholz, Darmstadt (DE)

(73) Assignee: Merck Patent GmbH, Darmstadt (DE)

(21) Appl. No.: 14/000,408

(22) PCT Filed: Feb. 3, 2012
(86) PCT No.: PCT/EP12/00490

§ 371 Date: Aug. 20, 2013

(30) Foreign Application Priority Data
Feb. 25, 2011 (EP) 11001570.8

(51) Int. Cl. A61K 31/198; A23L 1/305; A61K 45/06

(52) U.S. Cl. CPC A61K 31/198; A61K 45/06; A23L 1/3051
USPC 424/682; 514/561; 514/167; 514/54; 514/62

(57) ABSTRACT

The invention is directed to a composition for the use to increase joint stability or postural stability, wherein said composition comprises branched chain amino acids. It is further directed to the use of branched chain amino acids for the preparation of a food supplement or medicament for the prophylaxis and/or treatment of joint and/or postural instability.

The invention is directed to a composition for the use to increase joint stability or postural stability, wherein said composition comprises branched chain amino acids. It is further directed to the use of branched chain amino acids for the preparation of either a food supplement or a medicament for the prophylaxis and/or treatment of joint and/or postural instability. In 2008 eight million people in Germany suffered from joint related diseases such as osteoarthritis (OA), rheumatoid arthritis (RA) and others.

360 000 had a joint replacement, therefrom the majority regarding the hips and the knees (Press release Endoprothetic clinic Ortenau (19.05.2010)). Joint replacements are normally the last option to relieve pain in damaged or arthritis joints and restore the range of motion available in joints.

Initially exercise, strengthening, and stretching techniques are performed to decrease joint pain and increase joint range of motion. However, if significant relief of pain and improvement in mobility is not achieved, a joint replacement is an important consideration to improve function of life and to enable people to be physical active.

According to the WHO physical inactivity is now identified as the fourth leading risk factor for global mortality (6% of death globally). Physical inactivity levels are rising in many countries with major implications for the prevalence of noncommunicable diseases (NCDs) and the general health of the population worldwide. (WHO Brochure “GLOBAL recommendations on PHYSICAL ACTIVITY FOR HEALTH” 2010).

It is obvious that joint pain and joint stiffness restrain people from being physical active. Furthermore the perceived loss of stability (frailty) increases the fear to tumble, people lose confidence for being physical active.

Joint stability is a pre-requisite of mobility and physical activity. Joint stability describes the displacement or respectively subluxation of two bones relative to each other and can be measured. A subluxation is the condition of a vertebra that has lost its proper juxtaposition with the one above or the one below or both to an extent less than a luxation, which impinges nerves and interferes with the transmission of mental impulses.

Preservation of joint stability cannot be ascribed to the ligaments alone, but should be considered as a synergistic function in which bones, joint capsules, ligaments, muscles, tendons, and sensory receptors and their spinal and cortical neural projects and connections function in harmony. Biomechanical mal-alignment of the joints can contribute to the development of OA.

Joint instability refers to a joints inability to withstand normal ranges of impact and motion without becoming injured, displaced or dislocated. Various musculoskeletal, or extra-articular, structures may be involved such as the bones, tendons, ligaments, muscles and soft tissue. Compromised intra-articular (within the cavity of the joint) integrity may also contribute to joint instability.

One of the main mechanical functions of articular cartilage is to act as a low-friction, load-bearing surface. These mechanical loads are absorbed by the cartilage extracellular matrix (ECM), where they are subsequently dissipated and transmitted to chondrocytes (cartilage cells). Due to its unique location at joint surfaces, articular cartilage experiences a range of static and dynamic forces that include shear, compression and tension.

Chondrocytes, cells specialised in the release of cartilage constituents, sense and convert the mechanical signals they receive into biochemical signals, which subsequently direct and mediate both anabolic (matrix building) and catabolic (matrix degrading) processes. These processes include the synthesis of matrix proteins (type II collagen and proteoglycans), proteases, protease inhibitors, transcription factors, cytokines and growth factors. Homoeostasis is strongly influenced by the type of loading, high strain rates which cause tissue damage, results in degradation and a decrease in matrix production as well as apoptosis.

A lack of joint stability leads to imbalanced mechanical loads of the cartilage and can thereby increase wear and tear. Osteoarthritis is characterised by a loss of cartilage.

Main causes for the development of OA are

Age

Obesity

Joint injury or joint overuse

Occupational stress: repetitive and monotonous movements at work and

Inactivity

Inactivity contributes to joint deterioration. Considering the fact that the cartilage in joints can only be nurtured by diffusion (due to a lack of blood vessels) and these diffusion processes are supported by the compression or flexion of the articular cartilage, the meaning of mobility is obvious.

Joint stability, especially knee stability is normally evaluated by the Lachmann test wherein the participant is asked to lie supine and the knee flexes around 30° (Torg J, Conrad W, Kalen V. Clinical diagnosis of anterior cruciate ligament instability in the athlete. Am J Sports Med 4: 84-93 (1976)). The examiner then stabilizes the femur and applies an anterior force on tibia without restraining axial rotation. Deflection of the tibiae against the patella is measured while the leg is charged with 75% of body weight and flexed 30°.

As described above physical activity can be used to increase joint stability and postural instability. Further, chondroprotectives, such as glucosamine and chondroitin sulfate, are frequently used in order to diminish loss of cartilage especially in osteoarthritis. Although some studies confirm some beneficial effects in OA further therapeutic possibilities are highly needed, especially such that increase joint stability and postural instability.

Surprisingly it was found by the present invention that the regular intake of at least two branched chain amino acids (for at least two month) leads to an increase of joint stability and postural stability and can thereby increase the quality of life. Accordingly, the present invention is directed to a composition for the use to increase joint stability and/or postural stability, wherein said composition comprises at least two branched chain amino acids.

Advantageously, the intake comprises L-leucine together with at least one of the branched chain amino acids L-iso-leucine and L-valine, as, for example, L-leucine and L-iso-leucine or L-leucine and L-valine. Accordingly, one embodiment of the invention is directed to a composition for the use to increase joint stability and/or postural instability, wherein the composition comprises L-leucine and at least one of the branched chain amino acids L-isoleucine and L-valine.

Advantageously, the composition contains L-leucine in an amount from about 25 to about 80% by weight, preferably from about 35 to about 75% by weight, more preferably from about 45 to about 70% by weight, most preferably about 64% by weight, based on the total weight of all branched chain amino acids. Therefore, the present invention is further directed to the composition for the use to increase joint stability or postural stability, wherein L-leucine is present in an amount from about 25 to about 80% by weight, preferably from about 35 to about 75% by weight, more preferably from about 45 to about 70% by weight, most preferably about 64% by weight, based on the total weight of all branched chain amino acids.

According to a further preferred embodiment the invention is directed to a composition for the use to increase joint stability or postural stability, wherein L-leucine is present in an amount from about 35 to about 80% by weight, L-iso-leucine in an amount from about 10 to about 30% by weight and L-valine in an amount from about 10 to about 30% by weight.

According to an especially preferred embodiment the invention is directed to the composition for the use to increase joint stability or postural stability, wherein L-leucine, L-iso-leucine and L-valine are present in the weight ratio of about 2-6:0.5-1.5:0.5-1.5, preferably in a weight ratio of 3-5:0.75-1.25:0.75-1.25, more preferably in the weight ratio of about 3.5: about 1: about 1.

According to another preferred embodiment of the invention the composition of the invention may contain one or more further active ingredients. Therefore, the invention is also directed to a composition for the use to increase joint stability or postural stability, wherein the composition contains one or more further active ingredients.

In principle the active ingredient, which may be present in the composition, can be any pharmaceutical drug or any other ingredient, which after intake into the human body has a beneficial effect as, for example, vitamins, mineral substances, trace elements, roughage, enzymes or plant extracts. Preferred active ingredients, which may be present in the composition, are pain relieving substances, chondroprotectives, vitamins, plant extracts and/or mineral substances.

A preferred vitamin is vitamin D, preferred mineral substances are inorganic or organic calcium and/or magnesium salts, which are suitable for consumption, preferably in the form of carbonates, bicarbonates, phosphates, biphosphates, sulfates, bisulfates, chlorides, fluorides, citrates and/or lactates, preferred chondroprotectives are hyaluronic acid and/or glucosamine or its derivatives, such as chondroitin, and/or their salts, such as sulfates or hydrochlorides.

Accordingly, the invention is further directed to a composition for the use to increase joint stability or postural stability, wherein the active ingredients are vitamins, such as vitamin D, mineral substances, such as magnesium or calcium salts and/or chondroprotectors, such as hyaluronic acid and/or glucosamine or its derivatives, such as chondroitin, and/or their salts, such as sulfates or hydrochlorides.

Preferred plant extract are plants extracts, which are well known for their anti-inflammatory effect, as, for example, extracts from Boswellia serrata or Harpagophytum procumbens.

In principle the composition can be administered by all suitable routes, including but not limited to the oral and parenteral application route. However, oral application route is preferred. Therefore, a preferred embodiment of the invention is directed to the composition for the use to increase the joint stability or postural stability, wherein the composition is for oral use.

As the composition can be taken in various forms the presentation of the composition is not limited to any specific form. Examples of suitable forms for the intake of the composition are dietary means, such as dietary supplements, food products, such as a medical or functional food or a beverage product, e.g. as a complete meal, as part of a meal, as food additive or as powder for dissolution, or pharmaceutical formulations, e.g. in form of a tablet, a sachet or a capsule. Preferably the composition of the present invention is part of a food product, a dietary supplement and/or a pharmaceutical formulation. Hence, the present invention is further directed to a composition for the use to increase joint stability or postural stability, wherein the composition is in the form of a food product, a dietary supplement or a pharmaceutical preparation.

The terms “functional food” and “medical food” are understood to be any food, which is enriched with active ingredients so that it has a health-promoting or disease-preventing property beyond the basic function of supplying a composition of nutrition. Functional food can be processed food, which is commercially prepared food designed for ease of consumption, as a well as “normal” food. Food enriched with the composition of the present invention is understood to be functional food or medical food, which can be used to increase joint stability and postural stability.

Dietary supplements and pharmaceutical formulations can be provided as solid, semisolid or liquid dosage forms. Examples of a solid dosage form are tablets, dragées, capsules, granules or powders, examples of semisolid dosage form are creams or gels, examples of liquid dosage forms are solutions or suspensions. Preferred are solid dosage forms such as tablets, dragées, capsules, granules or powders. Therefore, a preferred embodiment of the invention is directed to a composition for the use to increase joint stability and postural instability, wherein the composition is in solid dosage form, preferably in the form of a tablet, a dragée, a capsule or a powder.

Solid oral dosage forms are generally known in the art, they can be prepared, for example, by means of conventional mixing, granulating, confectioning, dissolving or lyophilizing processes.

For example, compositions for oral administration are obtained by combining the active ingredients with solid carriers, optionally granulating a resulting mixture and processing the mixture or granules, if desired or necessary after the addition of suitable excipients, to form tablets or dragée cores. Granules may be also used itself without further processing.

Suitable physiologically acceptable auxiliaries are fillers, such as sugars, for example lactose, mannitol or sorbitol, cellulose preparations and/or calcium phosphates, for example tricalcium phosphate or calcium hydrogen phosphate, and also binders, such as starch pastes using, for example, corn, wheat, rice or potato starch, gelatin, tragacanth, methylcellulose and/or polyvinylpyrrolidone, and, if desired, disintegrators, such as the above-mentioned starches, and also carboxymethyl starch, cross-linked polyvinylpyrrolidone, agar, or alginic acid or a salt thereof, such as sodium alginate. In one aspect of the invention the compositions of the invention may be lactose-free. Further excipients may be especially flow-conditioners and lubricants, for example silicic acid, talc, stearic acid or salts thereof, such as magnesium or calcium stearate, and/or polyethylene glycol.

Dragée cores are provided with suitable coatings, there being used inter alia concentrated sugar solutions which may contain arabic gum, talc, polyvinylpyrrolidone, polyethylene glycol and/or titanium dioxide, or coating solutions in suitable organic solvents or solvent mixtures. Dyes or pigments may be added to the tablets or dragée coatings, for example for identification purposes or to indicate different doses of active ingredient.

Powders are prepared by comminuting the substances of the composition to a suitable fine size and mixing it with a physiologically acceptable auxiliary comminuted in a similar manner, such as, for example, an edible carbohydrate, such as, for example, starch or mannitol.

A flavour, preservative, dispersant and dye may likewise be present. Other preferred orally administrable solid dosage forms are capsules including hard and soft capsules, especially hard gelatin capsules, sealed capsules consisting of gelatin and a plasticizer, such as glycerol or sorbitol. The hard gelatin capsules may comprise the composition of the invention in the form of granules, for example in admixture with fillers, such as lactose, binders, such as starches, and/or glidants, such as talc or magnesium stearate, and, if desired, stabilizers. In soft capsules the composition of the invention is preferably dissolved or suspended in suitable liquids, such as fatty oils, paraffin oil or liquid polyethylene glycols, it is likewise being possible to add stabilizers.

Conventional additives may be included in the compositions of the invention, including any of those selected from preservatives, chelating agents, osmotic agents, buffers or agents for pH adjustment, effervescing agents, sweeteners, e.g. artificial sweeteners, flavoring agents, coloring agents, taste masking agents, acidulants, emulsifiers, stabilizers, thickening agents, suspending agents, dispersing or wetting agents, antioxidants, acidulants, texturizers, antifoams, and the like.

In addition to the foregoing the present invention also provides a process for the production of a composition, e.g. nutritional or pharmaceutical formulation, as hereinbefore defined, which process comprises bringing the individual components thereof into intimate admixture and, if required, compounding the obtained composition in a food or beverage product, for example ready-to-use drink, or in unit dosage form, for example filling said composition into hard capsules.

Advantageously the composition of the present invention is adapted to provide the branched chain amino acids in daily dosages in the range of from about 1000 to about 5000 mg L-leucine, from about 300 to about 2000 mg L-iso-leucine, and from about 300 to about 2000 mg L-valine, preferably from about 2000 to about 4000 mg L-leucine, from about 600 to about 1500 mg L-iso-leucine and from about 600 to about 1500 mg L-valine, and more preferably, about 3200 mg L-leucine, about 900 mg L-iso-leucine and about 900 mg L-valine.

To provide the daily dosage of branched chain amino acids the composition can be administered as one single dose, such as, for example, as one meal or one dosage form as defined herein, such as one solid dosage form like one tablet, one capsule or as one sachet, which contains the appropriate amount of granules or powders. Alternatively, the daily dosage can also be provided by distributing the daily dosage into or more dosage forms, such as, for example, two or more tablets, capsules and/or sachets.

According to a preferred embodiment of the invention the daily dosage is provided by a single composition, such as, for example, by one meal, tablet, capsule or sachet.

According to a preferred embodiment of the invention the daily dosage of branched chain amino acids is adapted to the body weight of the person taking the composition. Relating to the branched chain amino acid L-Leucine Table 1 discloses typical amounts of intake, which are preferably used depending on the body weight.

TABLE 1
preferred daily dosages of L-leucine

| | 30 mg kg⁻¹ | 40 mg kg⁻¹ | 50 mg kg⁻¹ | 60 mg kg⁻¹ |
|---|---|---|---|---|
| Body | body weight | body weight | body weight | body weight |
| weight [kg] | day⁻¹ | day⁻¹ | day⁻¹ | day⁻¹ |
| 50 | 1.5 g | 2 g | 2.5 g | 3.0 g |
| 60 | 1.8 g | 2.4 g | 3.0 g | 3.6 g |
| 70 | 2.1 g | 2.8 g | 3.5 g | 4.2 g |
| 80 | 2.4 g | 3.2 g | 4.0 g | 4.8 g |

The weight adapted daily dosages of L-leucine are preferably combined with further branched chain amino acids in weight ratios as identified above resulting in that the respective amount of L-leucine is combined with L-iso-leucine and L-valine in a weight ratio of 2-6:0.5-1.5:0.5-1.5, preferably in a weight ratio of 3-5:0.75-1.25:0.75-1.25, more preferably in a weight ratio of about 3.5: about 1: about 1.

According to another preferred embodiment of the invention the composition can be used for the prophylaxis and/or treatment of joint instability and/or postural instability. Hence the invention is also directed to a composition for the use of prophylaxis and/or treatment of joint instability and/or postural instability, wherein such composition comprises at least two branched chain amino acids. The composition for the use of prophylaxis and/or treatment of joint instability and/or postural instability can be any of the compositions described herein before.

Further, the invention is also directed to the use of at least two branched chain amino acids for the manufacture of a medicament for the prophylaxis and/or treatment of joint instability and/or postural instability.

The examples explain the invention without being restricted thereto.

### EXAMPLES

Except it is explicitly stated otherwise the amounts of ingredients disclosed hereinafter are given in milligram (mg).

### Powder Composition

Powder

| | | |
|---|---|---|
| L-Leucin | | 3200 |
| Iso-Leucin | | 900 |
| L-Valine | | 900 |
| Maltodextrin | 50-200 | |
| Opadry white | 10-100 | |
| Aspartame | 10-100 | |
| Flavours | 20-120 | |
| Sucroseester | 10-100 | |

### Production

1. Weighing
2. Sieving
3. Blending
4. Packaging

### Granules

Granules

| | | |
|---|---|---|
| L-Leucin | | 3200 |
| Iso-Leucin | | 900 |
| L-Valine | | 900 |
| Silicondioxid | 100-300 | |
| Citric acid | 2000-4000 | |
| Maltodextrin | 1000-3000 | |
| Lecithin | 10-100 | |

### Production

1. Weighing
2. Sieving
3. Blending
4. Granulation (e.g. fluid bed granulatuion, wet granulation)
5. Blending
6. Packaging

### Milk Powder (Nutritional Composition)

Milk Powder

| | | |
|---|---|---|
| L-Leucin | | 3200 |
| Iso-Leucin | | 900 |
| L-Valine | | 900 |
| Silicondioxid | 100-300 | |
| Milk powder | 10000-250000 | |

### Production

1. Weighing
2. Sieving
3. Blending
4. Packaging

### Measurement of Joint Stability

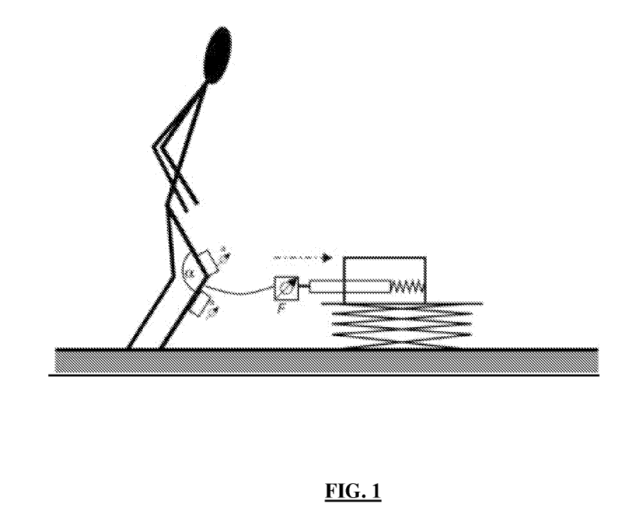

Knee stability after regular intake of BCAA is evaluated by using a further development of the Lachmann test. A device with a high temporal and local resolution for the measurement of the tibial displacement under functional conditions has been developed at the University of Stuttgart. Basis of the measurement is the determination of the deflection of the tibiae against the patella vie an 10 g accelerometer at dynami force application of two different forces. The leg is charged with 75% of the body weight (control via scales) and flexed 30° (FIG. 1).

### Measurement of Postural Stability

Postural stability (total, anteroposterios, and mediolateral postural stability) is assessed according to the balance test as described by Myer et al. (Myer G, Brunner H, Meldon P, Peterno M, Ford K, and Hewett T Specialised neuromuscular training to improve neuromuscular function and biomechanics in a patient with quiescent juvenile rheumatoid arthritis Phys Ther 85:791-802 (2005)). In such method a stabilometer Balance-coordination system (for example GK 1000 IMM Elektronik GmbH) is used to measure the sway amplitude of the center of pressure (COP) within a 40 sec bipedal standing test. Person has to stand with both legs on an instable platform and then all the movements to regain balance can be measured electronically and thereby the postural stability.

### Study

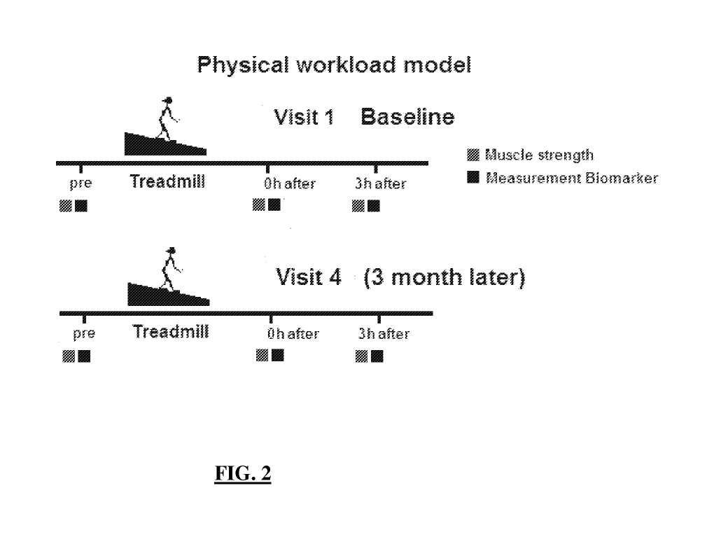

The effect of the composition of the present invention on joint stability was tested in a double-blind randomized placebo controlled clinical study. 48 healthy subjects, aged 54 to 72 years with a BMI from 22 to 30 (ratio men and women 1:1) and with low activity level were included in the current trial. In total 45 subjects finished the main part of the study. Participants had to exercise moderately 3 times the week for 30 minutes using a device which allows to train with the individual body weight and to increase the efforts by increased repetition. During the study (3 months) participants had to take branched chain amino acid containing powder composition as described above (verum) or placebo once per day closed to the highest level of physical activity. Baseline values for physical performance and biomarkers of cartilage synthesis and degradation were evaluated at the beginning of the study (visit 1) and further measurement occurred intermittently every month, ending with the end of the study (visit 4). Measurements were done before (pre), directly after (0 h after) and three hours after (3 h after) having a specified physical work-load (see FIG. 2).

Physical work-load was exercised by using a “Physical workload model”. In such Physical workload model participants had to perform an eccentric walk down with a 25% gradient on a treadmill. Subjects were additionally loaded with 10% of their body weight in the form of a lead-weighted jacket. Women had to walk at 4 km/h, men at 5 km/h. At visit 1 participants were asked to walk as long as possible according to their individual capacity without exceeding 40 minutes. At visit 4 the individual protocol from visit 1 was repeated. Before treadmill (pre) and directly after treadmill (0 h after) and at 3 hours (3 h after) leg extension was assessed using the David Back concept (David Health Solutions LTD) (Wydra G. Zur Problematik von Normen in der Bewegungstherapie Krankengymnastik 2004) The measurement of leg extension represents a validated method to evaluate the muscle strength and thereby also loss of strength (Saris W. et al, PASSCLAIM—Physical performance and fitness Eur J Nutr 2003).

As biomarkers for cartilage synthesis and degradation CP2 (C-propeptide of type two collagen CP2, synonym CII CP), a marker reflecting the synthesis of new collagen, and C2C (type two collagen collagenase cleavage neoepitop), a marker reflecting the degradation of collagen from blood samples, were assessed (Bijlsma J et al Osteoarthritis: an update with relevance for clinical practice Lancet 2011). The rationale underlying the use of such biomarkers is that physical workload of joints leads to the release of structural fragments of collagen in the cartilage which have to be replaced by the synthesis of new collagen. Such increased metabolism induced by physical workload results in a decrease of the ratio of cartilage degradation and synthesis biomarkers (C2C/CP2). Therefore, a decline of the workload induced decrease of C2C/CP2 ratio can be interpreted as an improvement of joint stability.

### Results

Under physical strain on the joints (in the Physical workload model) the intake of the verum led to an additional improvement of joint stability compared to placebo.

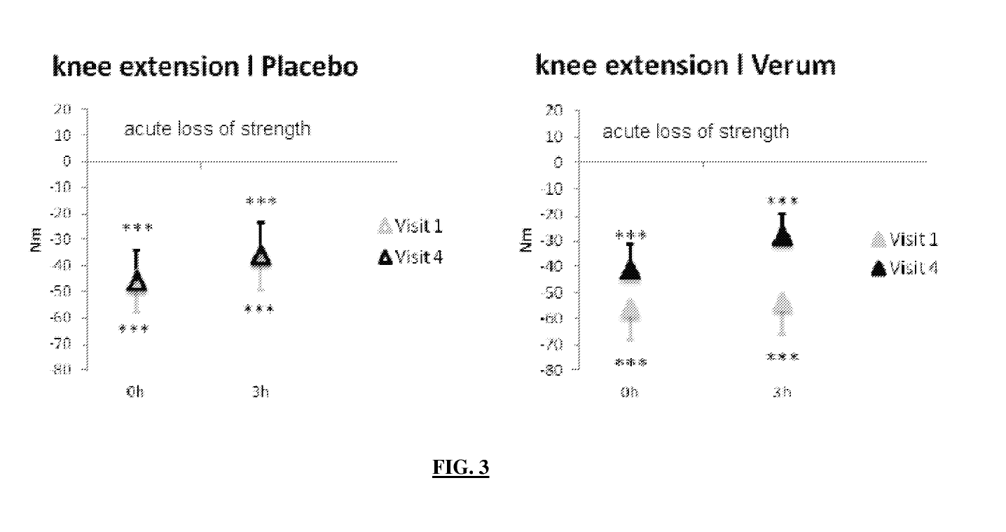

The results on strength loss after physical strain are presented in FIG. 3. As shown by this the loss of the strength of the physical strain was significantly less pronounced in the verum group after taking verum for 3 month as compared to the placebo group, wherein the loss of strength was nearly identical at the two time points. The less pronounced loss of strength in the verum group indicates its beneficial increase in strength endurance. The increase of strength endurance is beneficial in daily activities such as stair climbing and long distance walking and might encourage especially older people to stay physically active. Physical activity is necessary to maintain healthy joints.

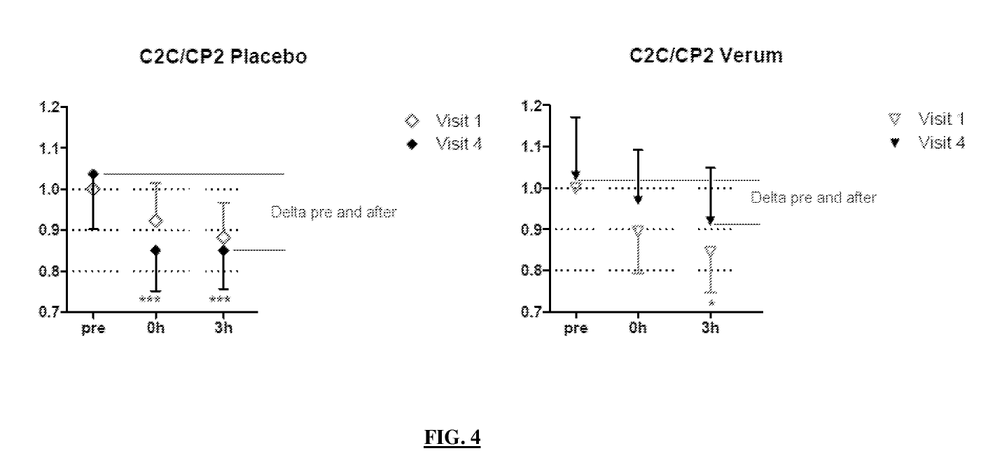

The results on ratio of cartilage degradation and synthesis biomarkers of the physical strain are presented in FIG. 4. As shown by this the decrease of the ratio of cartilage degradation and synthesis biomarkers (C2C/CP2) is less pronounced in the verum group as indicated by the Delta between the values prior physical strain (pre) and of the values directly after (0 h) and three hours after (3 h) physical strain. Further, after three months intake of study medication (visit 4) the verum group shows a less pronounced decline of the C2C/CP2 ratio zero (0 h) and three hours after (3 h) physical strain compared to the beginning (visit 1), which is not the case in the placebo group.

Taken together these results clearly demonstrates that treatment with the composition containing at least two branched chain amino acids results in a reduction of strain on joint structures induced by physical load and an improvement of the joint stability.

1. Composition for the use to increase joint stability and/or postural stability, wherein said composition comprises at least two branched chain amino acid.

2. Composition according to claim 1, wherein the composition comprises L-leucine and at least one of the branched chain amino acids L-isoleucine and L-valine.

3. Composition according to claim 1, wherein L-leucine is present in an amount from about 25 to about 80% by weight, preferably from about 35 to about 75% by weight, more preferably from about 45 to about 70% by weight, most preferably about 64% by weight, based on the total weight of all branched chain amino acids.

4. Composition according to claim 1, wherein L-leucine present in an amount in an amount from about 35 to about 80% by weight, L-iso-leucine in an amount from about 10 to about 30% by weight and L-valine in an amount from about 10 to about 30% by weight.

5. Composition according to claim 1, wherein L-leucine, L-iso-leucine and L-valine are present in the weight ratio of 2-6:0.5-1.5:0.5-1.5, preferably in a weight ratio of 3-5:0.75-1.25:0.75-1.25, more preferably in a weight ratio of about 3.5: about 1: about 1.

6. Composition according to claim 1, wherein the composition contains one or more further active ingredients.

7. Composition according to claim 6, wherein the active ingredients are vitamins, such as vitamin D, mineral substances, such as magnesium or calcium salts and/or chondroprotectors, such as hyaluronic acid and/or glucosamine or its derivatives, such as chondroitin, and/or their salts, such as sulfates or hydrochlorides.

8. Composition according to claim 1, wherein the composition is for oral use.

9. Composition according to claim 1, wherein the composition is adapted to provide the branched chain amino acids in daily dosages in the range of from about 1000 to about 5000 mg L-leucine, from about 300 to about 2000 mg L-iso-leucine, and from about 300 to about 2000 mg L-valine, preferably from about 2000 to about 4000 mg L-leucine, from about 600 to about 1500 mg L-iso-leucine and from about 600 to about 1500 mg L-valine, and more preferably, about 3200 mg L-leucine, about 900 mg L-iso-leucine and about 900 mg L-valine.

10. Composition according to claim 1, wherein the composition is in the form of a food product, a dietary supplement or a pharmaceutical preparation.

11. Composition according to claim 10, wherein the composition is in the form of a solid dosage form, preferably in the form of a tablet, a capsule, a granule or a powder.

12. Composition for the use of prophylaxis and/or treatment of joint instability and/or postural instability, wherein such composition comprises at least two branched chain amino acids.

13. Composition according to claim 12, wherein the composition comprises L-leucine and at least one of the branched chain amino acids L-isoleucine and L-valine.

14. A method for the manufacture of a medicament for the prophylaxis and/or treatment of joint instability and/or postural instability which comprises providing at least two branched chain amino acids together in a composition.

15. Method according to claim 14, wherein the medicament comprises L-leucine and at least one of the branched chain amino acids L-isoleucine and L-valine.
